(19) United States
(12) Patent Application Publication
(10) Pub. No.: US 2008/0057561 A1
(43) Pub. Date: Mar. 6, 2008

(54) CELL CULTURE TREATMENT APPARATUS AND CELL CULTURE TREATMENT METHOD

(75) Inventors: Atsushi Takahashi, Kawasaki-shi (JP); Tsutomu Honma, Fuchu-shi (JP); Takahisa Ibii, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/843,564

(22) Filed: Aug. 22, 2007

(30) Foreign Application Priority Data
Aug. 29, 2006 (JP) 2006-232178

(51) Int. Cl. C12N 1/00; C12M 1/12

(52) U.S. Cl. 435/243; 435/297.1; 435/297.2

(57) ABSTRACT

The present invention provides a cell culture treatment device for culturing, treating and collecting cells, which includes a flow path, a cell-retaining section, a solution inlet, a solution outlet, a cell collection port and a lid. The present invention also provides an device for easily, efficiently separating and collecting the cells thereby in a short period of time, and provides a method therefor.

### BACKGROUND OF THE INVENTION

1. Field of the Invention

The present invention relates to a cell culture treatment device which enables cells to be collected after having been subjected to at least one process of a cell culture and treatment with a chemical agent in a flow path, and has a cell-retaining section made from a porous material; and also relates to a cell culture treatment method with the use of the device. More specifically, the present invention relates to the cell culture treatment device provided with the cell-retaining section and a specifically designed outlet for collecting cells in the flow path, and relates to the cell culture treatment method including changing a direction of a passing solution with respect to the cell-retaining section.

2. Description of the Related Art

Recently, a biomicrochip (biochip) has been actively studied and developed which employs a biological substance such as a nucleic acid, an enzyme, an antibody and a cell as a functional element, by amalgamating a biotechnology and a micromachining technology. When producing the biochip, an object to be measured can be concentrated in a small region or a small amount of the object can be measured by using the micromachining technology cultivated in a semiconductor production process. As a result, a significant effect can be obtained due to the small scale.

Various types of biochips and microchips are discussed in detail in “Micro Kagaku-chip No Gijutsu-to-Ohyou (Technology and Application of Micro Chemical Chip)”: Maruzen Co. LTD., and “Microfluidic Technology and Applications”: Research Studies Press LTD. A technical field particularly receives attention, which targets at a cell that involves a higher level of a life phenomenon than the above described biological substances. Against this backdrop, various devices have been proposed which aim at culturing a cell or measuring a substance in a microchip.

Japanese Patent Application Laid-Open No. 2003-294741 discloses an device which can analyze a function of a living cell by arranging a cell culture section and a section for detecting a response from the cell in a microchip. In addition, Japanese Patent Application Laid-Open No. 2005-253412 proposes a microwell array chip which has a microwell structure made from polydimethylsiloxane (PDMS) and a sensor section containing a pH-responsive fluorescent dye prepared in the bottom of the well, and can evaluate the activity of a cell.

On the other hand, as for a microchip having a porous material in a flow path, the following technologies have been proposed so far. Apparatuses are proposed in Sensors and Actuators (A) (Physical, vol. 73, pp. 184-191 (1999)) and Sensors and Actuators (B) (Chemical, Vol. 67, pp. 203-208 (2000)), which have a filter formed of a base material made from silicon in the flow path by using a micromachining technology, and are directed at treating particles. These devices aim at making the particles retained on the filter arranged in the flow path, and chemically coated or etched with a reagent in the state. The devices collect the desired particles from an inlet side by regurgitating a supplied liquid in a step of collecting the treated particles.

There is also Japanese Patent Application Laid-Open No. 2004-317128 which discloses a method for forming a porous material in a fine channel. Specifically, this is a method for forming the porous material made from a polymer so as to traverse the fine channel in a horizontal direction, by photo-curing the polymer by causing a sol-gel reaction, a crosslinking reaction or the like while irradiating the polymer with light. This technology aims at making cells bonded and immobilized on the porous material, by hydrophilizing and hydrophobizing the surface of the porous material or immobilizing a catalytic component, an enzyme, an antibody, an antigen or the like. In other words, the above described device aims at capturing an object such as a desired cell on a filter portion to measure characteristics of the object.

### SUMMARY OF THE INVENTION

However, the device according to each of the above described bulletins has been directed at culturing cells or detecting a cell and have not been composed so as to collect the cells after having had cultured the cells or detected the cell. Because of this, the cells once taken in the device remain in a state of being fixed to the device, so that the cells and the device are hardly reused. Particularly, the tendency has been remarkable when a free-floating cell is cultured, a cellular aggregate (spheroid) is circulation-cultured, and an adhesive cell group is cultured. In other words, the devices have not aimed at collecting an object such as a cell after having treated the object with a chemical agent or the like, and accordingly have had a different structure from the structure according to the present invention.

In addition, the above devices described in Sensors and Actuators (A) : Physical, vol. 73, pp. 184-191 (1999) and Sensors and Actuators (B): Chemical, Vol. 67, pp. 203-208 (2000) have aimed at treating and collecting particles, but not at culturing and collecting cells. Furthermore, the devices have collected the particles from an inlet side by regurgitating the flow of a solution when collecting the particles. Because of this, when living cells are collected by using the devices, they have recontacted a treatment solution or the like, so that there has been a case in which the cells cannot be reused after having been collected, because the cells have changed the activity or died.

For this reason, an device has been desired to be developed which can further reduce the possibility of contamination due to such recontact. The present invention is designed at solving such a problem, and is directed at providing a cell culture treatment device for culturing, treating and collecting cells, and providing a cell culture treatment method with the use of the device. Specifically, the present invention relates to the cell culture treatment device having a cell-retaining section which aims at holding cells in a flow path.

The device can simply and efficiently collect cells which have been cultured for a desired period of time or have been treated in various ways, in a short time. The device can also trap the cells in its flow path because of having the cell-retaining section such as a porous material arranged in the flow path. Furthermore, the device has a structure of the porous material arranged in the flow path so as to traverse the flow path at a predetermined angle with respect to a flow direction of a solution, preferably in a vertical direction to the flow direction, and accordingly can inhibit the trapped cells from being stacked due to the weight of the cells. As a result of this, the device can efficiently treat the cells, can efficiently exchange a culture solution, and can reduce cell death (necrosis) due to a deficiency of oxygen and nutrients.

In order to solve the above described problems, the present invention provides the following devices and methods.

The present invention is directed to a cell culture treatment device for culturing, treating and collecting cells, comprising:

a flow path for passing a cell suspension, a chemical solution and a buffer solution;

a cell-retaining section which can retain cells and is placed in the flow path;

a solution inlet connected to a first end of the flow path;

a solution outlet connected to a second end of the flow path;

a cell collection port connected to the flow path in between the cell-retaining section in the flow path and the first end; and

a lid capable of opening/closing the cell collection port.

The cell-retaining section can be made from a porous material.

The flow path can include a flow path which extends in a vertical direction, and the porous material is arranged in the vertical flow path so that the plane direction can be parallel to the bottom part of the flow path.

The porous material can have an average pore size smaller than a size of a single cell.

The average pore size of the porous material can be 1 μm or smaller.

The cell culture treatment device can further comprise an opening/closing section for the flow path, which can open/close the flow path and is arranged at a portion in the flow path between the first end and a part at which the cell collection port is connected to the flow path.

The cell culture treatment device can further comprise a liquid-sending unit which can send a solution and is connected to at least one of the solution inlet and the solution outlet.

The present invention is directed to a cell culture treatment method with the use of the cell culture treatment device comprises the steps of:

passing a cell suspension containing the cells from the solution inlet to the solution outlet with the cell collection port closed with the lid to make the cell-retaining section retain cells,

performing at least one of the culture of the cells and the treatment of the cells after the retaining by passing from the solution inlet to the solution outlet at least one solution selected from the group consisting of a cell culture medium, a chemical solution for treating the cells and a buffer solution in the state of making the cell-retaining section retain the cells; and

flowing a solution from the solution outlet to the cell collection port after the performing step with the cell collection port opened with the lid to liberate the cells which have been retained in the cell-retaining section and collect at the cell collection port the cells.

The present invention is directed to a cell culture treatment method with the use of the cell culture treatment device comprises the steps of:

passing a cell suspension containing the cells from the solution inlet to the solution outlet with the cell collection port closed with the lid and the opening/closing section opened to make the cell-retaining section retain cells,

performing at least one of the culture of the cells and the treatment of the cells after the retaining by passing from the solution inlet to the solution outlet at least one solution selected from the group consisting of a cell culture medium, a chemical solution for treating the cells and a buffer solution in the state of making the cell-retaining section retain the cells; and

flowing a solution from the solution outlet to the cell collection port after the performing step with the cell collection port opened and the opening/closing section path closed with the lid to liberate the cells which have been retained in the cell-retaining section and collect at the cell collection port the cells.

As described above, the flow path has at least two ends. The flow path is connected to a solution inlet at “a first end” out of the ends, and is connected to a solution outlet at “a second end”. The first end and the second end are connected to the solution inlet and the solution outlet respectively at positions having the same height or positions having different heights. For instance, in FIG. 2B, reference numeral 22 forms the first end, and reference numeral 23 forms the second end. In addition, a “solution” in the present specification represents a cell suspension, a chemical solution or a buffer solution.

Further features of the present invention will become apparent from the following description of exemplary embodiments with reference to the attached drawings.

### BRIEF DESCRIPTION OF THE DRAWINGS

FIG. 1 is a top plan view illustrating an example of a cell culture treatment device according to the present invention.

FIGS. 2A and 2B are sectional views of a cell culture treatment device sectioned along the line 2A-2A of FIG. 1.

FIG. 3A is a schematic block diagram illustrating an example showing a state of cells which are captured by a cell-retaining section in a cell culture treatment device according to the present invention.

FIG. 3B is a schematic block diagram illustrating an example showing a state of cells when they are collected from a cell-retaining section in a cell culture treatment device according to the present invention.

FIG. 4 is a schematic block diagram illustrating an example of a porous material according to the present invention.

FIG. 5 is a top plan view illustrating a cell culture treatment device according to Example 1.

FIGS. 6A, 6B, 6C, 6D and 6E are developed top plan views of a cell culture treatment device according to Example 1.

FIGS. 7A and 7B are perspective views illustrating an example of a method for using a cell culture treatment device according to Example 1.

FIG. 8 is a top plan view illustrating a cell culture treatment device according to Example 2.

FIG. 9 is a top plan view illustrating a cell culture treatment device according to Example 3.

FIG. 10 is a top plan view illustrating a cell culture treatment device according to Example 4.

FIG. 11 is a perspective view illustrating an example of a method for using a cell culture treatment device according to Example 4.

FIG. 12 is a perspective view illustrating an example of a method for using a cell culture treatment device according to Example 4.

FIG. 13 is a perspective view illustrating an example of a method for using a cell culture treatment device according to Example 4.

FIG. 14 is a perspective view illustrating an example of a method for using a cell culture treatment device according to Example 5.

FIG. 15 is a perspective view illustrating an example of a method for using a cell culture treatment device according to Example 5.

FIG. 16 is a perspective view illustrating an example of a method for using a cell culture treatment device according to Example 5.

### DESCRIPTION OF THE EMBODIMENTS

A cell culture treatment device according to the present invention has a trap (cell-retaining section) aiming at holding (retaining) cells in a flow path. By holding the cells in a desired region of the flow path as described above, the device according to the present invention can achieve a cell culture/treatment process while controlling the number of the cells, with a high degree of reproducibility and efficiency. The cell culture treatment device according to the present invention includes collecting the cells again which having been cultured or treated with a chemical agent or the like for a desired period of time, and then, having been held in the cell-retaining section. The cell culture treatment device can be also applied to a process of culturing cells while passing a cell culture medium through the flow path.

1. Cell Culture Treatment

“Culture or treatment for cells” described in the present specification and claims means that at least one step of the culture and treatment (cell by a medicament and the like) for the cells is carried out. Similarly, “culture, treatment and collection for cells” described in the present specification and claims means that any of the steps of the culture and collection for the cells, the steps of the treatment and collection for the cells or the steps of the culture, treatment and collection for the cells are carried out.

(Cell Treatment Step)

A step of treating cells includes a step of giving an influence on a function of the cell, by giving chemical stimulation or the like to the cell, for instance, with the use of a chemical agent. Incidentally, the chemical agent for giving “chemical stimulation with the use of a chemical agent” has only to be a biologically active compound. The chemical agent can be selected, for instance, from among an antibiotic, an antiseptic, an enzyme inhibitor, an antipyretic, an antiphlogistic, a growth factor, an antiproliferative factor, a tranquilizer, a cytokine, a hormone, a steroid, an estrogen and an enzyme.

In addition, a cell culture treatment device according to the present invention can be used for treating cell in vitro. The cell culture treatment device can be also used, for instance, as an device for a cell function evaluation test including the evaluation of a cell function, for creating a functional cell, for concentrating a useful cell, and for acquiring a function-modified transgenic cell.

The above described “functional cell” means a somatic cell which composes a living body, such as a hepatic cell and a nerve cell. A testing process for evaluating the cell function includes the steps of: trapping a cell group on the surface of a cell-retaining section such as a porous material; stimulating the cells with a liquid flow or a chemical agent; and measuring a response from the cell against the stimulus by using a well-known technique. Specifically, a method of measuring an active state of cells from an amount of signals which change depending on the cell function can be applied. In addition, a method (fluorescence method) can be applied which measures fluorescence intensity that varies after having made cells absorb fluorescent substances and stimulated the cells, or before and after the stimulus. Alternatively, a chemiluminescent method or an electrochemical method can be applied.

The cell culture treatment device also can avoid cells from being contaminated by recontacting a treatment solution or the like in a stage of collecting the cells which has been subjected to a desirable treatment for a desired period of time, by collecting the cells from a cell collection port that is installed in a different position from a solution inlet.

(Cell Culture Step)

The cell culture treatment device also effectively cultures cells in a step of culturing cells, by passing a suitable culture medium for culturing the cells in the state of having retained the cells in a cell-retaining section, and thereby continuously bringing the cells into contact with the fresh culture medium. A type of the cell culture medium and the composition can be appropriately selected in accordance with the type of the cell.

A cell type to be used in the present invention is arbitrarily selected from among a cell derived from human or plant and animal, a cell group derived from human or plant and animal, a tissue derived from human or plant and animal, an aggregate derived from human or plant and animal, a bacterium, a protozoan, a yeast and a transgenic cell thereof. The cell culture treatment device is preferably applied to the culture and treatment of cells, which are difficult to be realized in a conventional static culture with the use of a culture flask.

Specifically, the cell culture treatment device can be applied to such a special culture as follows:

(1) culture for a free-floating cell group;

(2) culture for a cellular aggregate (spheroid) of a parenchymal cell group;

(3) continuous circumfusion culture for obtaining a useful product such as various lymphokines; and

(4) culture for a cell group having a high degree of chemotaxis.

The above item (1) relates to culture for a free-floating cell group. The above described “free-floating cell” means a cell group which does not need a substrate for bonding thereon in order to develop a basic breeding function, though being capable of weakly bonding to a substrate surface. More specifically, such a cell includes, for instance, a blood cell, a lymphocyte, a hybridoma and a protoplast. The free-floating cell can be statically cultured by using a culture flask, but has been hardly cultured in high density with the culture method. However, the free-floating cell can be efficiently perfusion-cultured in high density and in a microscale, by using a cell culture treatment device according to the present invention, because a cell-retaining section can substantially immobilize the free-floating cell group thereon.

The above described item (2) relates to culture for a cellular aggregate (spheroid) by using parenchymal cells. The above described “parenchymal cell” means a cell which shoulders the most important function in objective organs and tissues, such as a hepatic cell in a hepar. More specifically, such a cell includes, for instance, a cell in the group including a hepatic cell, a beta cell of pancreas, a myocardial cell, a skin epidermal cell, a cartilage cell, a bone cell and a stem cell. Incidentally, the parenchymal cell is considered synonymous with a functional cell.

It has been known that a hepatic cell, for instance, is greatly damaged when detached from a surface of a base material for a subculture, because the hepatic cell has a high degree of bonding dependency. Because of this, it has been extremely difficult to culture such a cell with a usual static method.

On the other hand, in order to make a hepatic cell acquire a function of albumin production, which is known as the representative hepatocellular function, and the activity of cytochrome P450 that is a chemical metabolic enzyme system, three-dimensional culture is reported to be extremely effective which aggregates cells to a certain number (about several hundreds). Here, it is a useful factor for realizing the three-dimensional culture to culture the cell in a state in which nourishment and oxygen can be efficiently supplied and a waste product can be removed on the surface that can control the adhesion of the cell. Against this backdrop, a cell culture treatment device according to the present invention can efficiently stir and culture the cells by continuously culturing a cell aggregation in a cell-retaining section in a micro flow path while retaining the cells in the cell-retaining section and passing a culture medium therethrough, and also can collect the cells without damaging them.

The above described item (3) relates to matter production mainly using an animal cell as a main target. The matter production is specifically a production of a biomedicine, and the cell culture treatment device is used for producing various lymphokines, glycoproteins and antibodies. The biomedicine includes, for instance, erythropoietin and G-CSF (granulocyte colony-stimulating factor). A culture method for making cells produce the matter is broadly classified into static culture and suspension culture. Here, the static culture generally means a culture method of culturing cells while bonding cells on the bottom face with the use of a dish or a culture bottle. On the other hand, the suspension culture is a method of culturing cells by mechanically stirring the suspension with the use of a magnetic stirrer or an impeller immersed in a Sakaguchi flask or an incubator.

However, the productivity of the matter in the static culture has been extremely low in some cases when the culture condition is not set at an optimal condition from the viewpoint of the productivity. In addition, the productivity of the matter in the suspension culture has been lowered in some cases, because a high shearing stress applied to cells due to stirring and kills a large quantity of the cells. In contrast to this, a cell culture treatment device according to the present invention can prevent cells from being killed, by making the cells produce the matter while making a cell-retaining section such as a porous material retain the cells and passing a culture medium to the cells and simultaneously culture the cells on a suitable condition for the matter production; and as a result, can achieve a high degree of the matter productivity.

In the above process, a cell type to be used for the matter production can be appropriately selected from the group including Escherichia coli, a yeast and an animal cell. The animal cell includes, for instance, a Chinese hamster ovary cell (CHO cell), a PER.C6 cell, a BHK cell, an NSO cell, a HepG2 cell, a hybridoma and an insect cell strain. The animal cell is generally considered to produce a smaller amount of the matter than the Escherichia coli or the yeast, but when using mammalian cells, there is a characteristic technique including a technique of using complicated post-translational modification.

The above described item (4) relates to a method for culturing a cell group having a high degree of chemotaxis (mobility). The cell group specifically includes a coliform group. Because Escherichia coli generally has an extremely high degree of chemotaxis, it has been extremely difficult to measure Escherichia coli or to efficiently introduce a gene into the Escherichia coli. For this reason, in order to culture the Escherichia coli in high density for the above purpose, there has been no other choice but to embedding-culture the Escherichia coli by trapping the Escherichia coli in a three-dimensional space of a hydrous gel such as collagen. However, a cell culture treatment device according to the present invention can be used for the efficient treatment of introducing the gene into Escherichia coli, because the device has a cell-retaining section such as a porous material and can trap the Escherichia coli in a desired space of the cell-retaining section.

2. Cell Culture Treatment Device

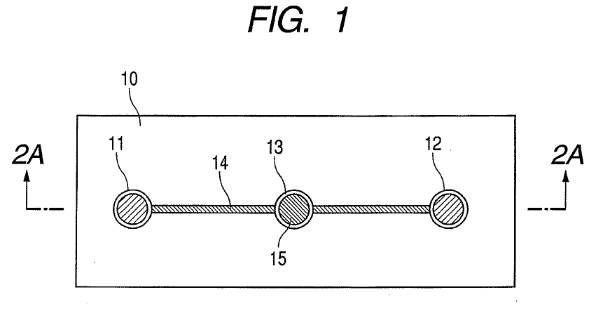

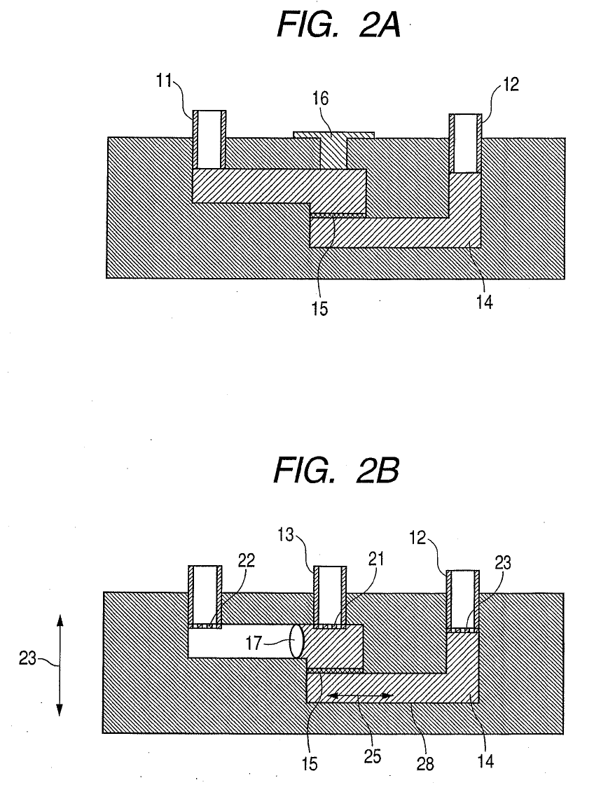

In the next place, an exemplary embodiment of a cell culture treatment device according to the present invention will be now described with reference to the attached drawings. The cell culture treatment device according to the present invention includes a flow path for flowing a solution and a porous material (cell-retaining section) for capturing cells. The flow path communicates with a solution inlet and a solution outlet through a first end and a second end respectively. The flow path further communicates with a cell collection port which can be opened/closed by operating a lid. FIG. 1 to FIGS. 2A and 2B illustrate an example of a schematic view of the cell culture treatment device according to the present invention.

This cell culture treatment device has one or more solution inlets for passing a solution (treatment liquid such as cell-containing liquid, culture medium and reagent) into the flow path and the solution outlet for discharging the solution outside the flow path, which are connected to the flow path. In addition, the cell culture treatment device has a space including the cell-retaining section and the cell collection port for collecting cells arranged in the flow path.

The number of solution inlets may be one or more, and may be two or more. When having two or more solution inlets, the cell culture treatment device can introduce a cell-containing liquid and a reagent from independent solution inlets. For instance, the cell culture treatment device can introduce a cell culture medium from one solution inlet and a reagent for bringing cells in contact with the reagent from the other solution inlet. Thus, the cell culture treatment device can be also used for a process of introducing the solutions (cell culture medium, cell-containing liquid and reagent) into a flow path, and mixing the solutions in the vicinity of the cell-retaining section to react them with each other. Furthermore, the cell culture treatment device can collectively adjust the concentration of reagents by arranging a plurality of admission ports for the reagents.

In addition, the cell culture treatment device may have one or more solution outlets and cell collection ports, and may have two or more of them. The cell collection port can be opened/closed by operating a lid, and can be turned into an opened state or a closed state by operating the lid, as needed. The lid includes a roof-shaped lid and a plug-shaped lid. But, the lid is not limited in particular, as long as it is such a member as to be able to prevent a liquid from leaking through the cell collection port even when having closed the cell collection port and having received a predetermined liquid pressure.

A cell culture treatment device according to the present invention has functions capable of performing a step of culturing and treating cells and a step of collecting the cells. The steps will be now described on the basis of an example illustrated in FIG. 1. At first, in the step of culturing and treating the cells, a solution is introduced into a container through an admission port (solution inlet) 11 as illustrated in FIG. 2A, by a liquid-sending device (liquid-sending unit) such as a pump, which is connected to the solution inlet 11. The solution flows in a flow path 14 via pores in a capturing mechanism (porous material) in the device, and is finally discharged from an exhaust port (solution outlet) 12. In the step, a cell collection port 13 is closed (made to be in a close state) with a lid 16 so as to prevent the contamination of the cells.

When a solution containing cells (cell-containing liquid) is passed from the solution inlet 11 to the solution outlet 12 at first as described above, the cells contained in the solution cannot flow in a cell-retaining section 15 because the cells are larger than a pore size in the cell-retaining section. The cells are also substantially immobilized on the cell-retaining section due to a pressure caused by the flow of the solution. Thus, the cell culture treatment device is prepared to culture or treat the cells in a state of having immobilized the cells, when a treatment liquid such as a cell culture medium or a reagent is passed to the solution outlet 12 from the solution inlet 11.

Next, in a step of collecting cells, a cell collection port 13 is opened (turned into an open state) by operating a lid, and a liquid is sent to the cell collection port 13 through a solution outlet 12 by a liquid-sending unit such as a pump connected to the solution outlet 12, as is illustrated in FIG. 2B. In the step, the cell collection port is connected to a flow path at a position between a first end and a cell-retaining section 15 (position 21 in FIG. 2B). Accordingly, the cells are liberated which have been retained in the cell-retaining section in the state illustrated in FIG. 2A, by reversing the flow of the solution in the above described way, and are collected through the cell collection port 13. In the above steps, a liquid may be sent from a solution inlet 11 to the solution outlet 12 by any of liquid-sending units connected to the solution inlet 11 and the solution outlet 12, and a liquid may be sent from the solution outlet 12 to the cell collection port 13 by any of them. Any of the liquid-sending units can be used by reversing a direction of sending the liquid.

A cell culture treatment device according to the present invention may have a valve (flow path opening/closing section) 17 for changing a flow direction of a liquid arranged in a flow path, so as to surely change the flow direction of the liquid according to the purpose. A generally reported valve mechanism can be appropriately used in the present invention as a valve mechanism for changing a flow path, which will be described below. In this case, the flow path opening/closing portion 17 is set at an opened state when a liquid is sent from a solution inlet 11 to a solution outlet 12, and the flow path opening/closing section 17 is set at a closed state when a liquid is sent from a solution outlet 12 to a cell collection port 13.

3. Each Section in Cell Culture Treatment Device

(Flow Path)

In a cell culture treatment device according to the present invention, a flow path composing the device can be formed by adhesively bonding or joining a plurality of substrates to each other. In other words, the flow path and a cell-retaining section can be formed of a plurality of the substrates. In one example for forming the flow path and the cell-retaining section by using a plurality of the substrates, the flow path is formed of grooves and through-holes which are formed in one or both facing planes of the substrate. In addition, the cell-retaining section is connected to the flow path, and is formed as a through-hole which penetrates one substrate. According to the method, the cell-retaining section can be easily prepared in the flow path, only by adhesively bonding or joining a porous material containing the through hole that composes the cell-retaining section, to the substrate that composes the upper and lower flow paths.

The flow path is constructed by overlapping the substrate having small pores penetrating the upper surface and under surface of the substrate with a substrate having a porous material. The substrate which composes the flow path can be formed by using an insulative solid substrate such as a material based on glass, silicon, quartz or silicon-based material, and plastics and polymers, for a base material. The base material more desirably has such optical transparency as to be capable of observing the inside with an invert microscope, and desirably has the surface of a substrate, which can be reformed by cleaning or pretreatment.

The substrate is cleaned by a wet cleaning method such as alkali cleaning, acid cleaning, water-based solvent cleaning, organic solvent cleaning and RCA cleaning, or a dry cleaning such as ultraviolet irradiation, ozone irradiation and oxygen plasma irradiation. In addition, when a slide glass, a quartz substrate or the like is used as a solid substrate, the surface of the substrate is reformed beforehand, for instance, by the steps of: cleaning the surface with any one selected from an acid, plasma, ozone, an organic solvent, a water-based solvent and a surface active agent; introducing a desired substituent into the surface through treatment such as silane coupling treatment; and controlling the free energy of the surface.

A shape and size of a flow path are not limited in particular, but can be adequately selected so as to match the type of a cell to be used and a quantity of the solution. The flow path also can include a vertical flow path which extends in a vertical direction, and a horizontal flow path which extends in a horizontal direction (a direction perpendicular to the vertical direction). The flow path can have the vertical flow path, and the vertical flow path can have a cell-retaining section of which the surface direction is horizontal (with respect to the bottom part of the flow path; or perpendicular to the vertical direction), in the vertical flow path. For example, in FIG. 2B, the flow path has a flow path that extends to a vertical direction 23, and the vertical flow path has the cell-retaining section 15 arranged therein. The cell-retaining section directs the plane in the horizontal direction 25 (that is, the cell-retaining section makes the plane direct in parallel to the bottom part 28 of the flow path).

(Cell-Retaining Section)

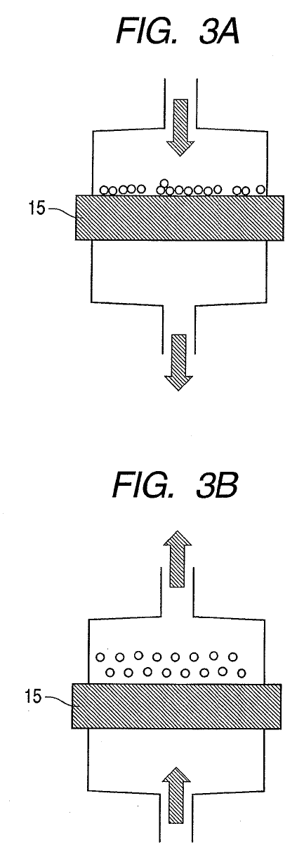

A cell culture treatment device according to the present invention makes cells captured in a flow path by a cell-retaining section 15 made from a porous material or the like. Specifically, when the cells are cultured or treated, they are retained in a desired region in the flow path by the porous material provided in the flow path, and further by a flow of a solution and the gravitation (FIG. 3A). When the cells are collected, they are carried to a cell collection port by the flow of the solution from a lower side (reverse direction) of the cell-retaining section made from the porous material or the like (FIG. 3B). The cell-retaining section 15 is arranged in the flow path so as to cover the whole section of the flow path. Specifically, the cell-retaining section is arranged so that all parts of the solution have to flow therethrough.

In one example of a cell culture treatment device according to the present invention, a porous material traps cells, which is arranged in a flow path so that the plane direction can be perpendicular to a flow direction of a solution. The surface of the porous material is formed so as not to make the cells adhere thereto, and thereby enables the cells after having been cultured to be easily collected.

The porous material can be prepared so as to promote or obstruct the adherence of cells onto the surface. The porous material can be also used for causing a reaction of cells by a negative interaction of preventing a cell or adhesive protein from non-specifically adsorbing to the surface.

In order to promote or obstruct the adherence of cells onto the surface of the porous material, specifically for instance, a flow rate of a solution is controlled, or the porous material is subjected to such pretreatment as not to make cells adhere to the pretreated surface. The above described “pretreatment” specifically means: a treatment for increasing the water repellency of the surface by coating the surface with a fluorine resin; a treatment of coating the surface with a blocking agent of extracellular matrix protein such as casein; and the like. An arbitrary method can be selected from the treatments.

The blocking agent is selected from among bovine serum albumin, casein, gelatine, skimmed milk, polyvinyl alcohol, polyvinylpyrrolidone, polyethylene glycol, phospholipid and a compound containing them. A surface active agent includes polyoxyethylene, octylphenyl ether, polyoxyethylene sorbitan monolaurate, polyoxyethylene alkylaryl ether phosphate, polyoxyethylene alkyl ether phosphate and polyoxyethylene alkylphenyl ether. A sugar includes saccharose, trehalose, heparin and low-molecular-weight heparin.

The content of the blocking agent in the above described treatment liquid is preferably 0.1 to 10 mass %. The content of the surface active agent in the above described treatment liquid is preferably 0.01 to 1 mass %. The content of the sugar in the above described treatment liquid is preferably 0.1 to 10 mass %. Furthermore, the porous material can be immersed in a solution containing the blocking agent, the surface active agent, and the sugar as needed, according to a widely-known method, be dried, and then be used. Thus treated porous material can show an adequate effect of preventing a protein not to be tested from non-specifically adsorbing onto the base material, facilitating an object to spread, and stably preserving a specifically bonded substance immobilized thereon.

As for a pore size and a void size of a porous material, such sizes are selected as to enable cells to be held (retained) by the porous material, and a culture medium and a buffer solution to pass through the porous material. In other words, the cell culture treatment device has such a structure as to be able to pass a liquid through the porous material arranged in a flow path, exchange the culture medium and a chemical agent for treatment and clean the cells with the buffer solution in the state of having held cells, and collect the cells very easily. Accordingly, the porous material has such a pore size and a void size as to enable the above operations.

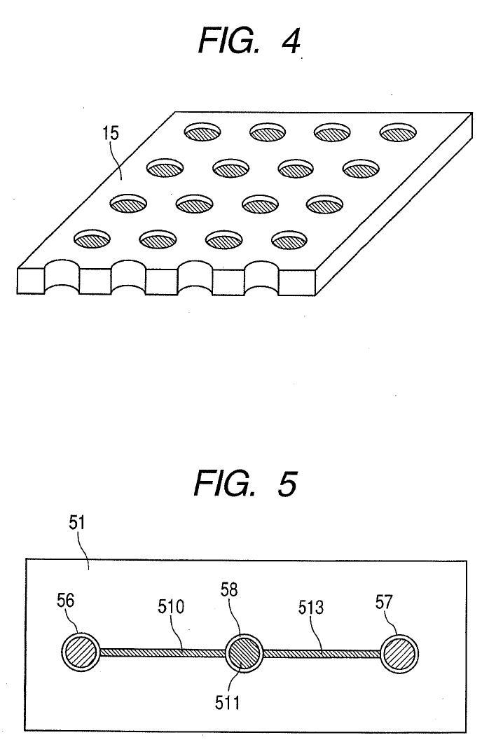

FIG. 4 illustrates one example of a form of a porous material 15 to be used as the cell-retaining section in the present invention. The shape of a pore of the porous material in the above example can be arbitrarily selected, as long as the pore meets an object of the present invention that the porous material captures cells but does not hinder the passage of a solution. The shape of the pore specifically includes a square, a rectangle, a circle, an oval and a triangle, but is not limited to the shapes in particular.

As for an average pore size of a porous material, the upper limit of a diameter can be about 1 μm when a pore or a void is circular, and the upper limit of a longer diameter (size of the longest part) can be about 1 μm when the pore or the void has another shape. The average pore size can be also smaller than a size of a single cell to be retained by the porous material. For instance, when the average pore size of the porous material has a scale equal to or smaller than a cell length, the pore of the porous material may be clogged with the cells, which may deteriorate the easiness of liquid exchange and cell collection of an advantage offered by the present invention.

A specific size of the pore in the porous material is optimally determined by the size of a test cell. For instance, suppose that the diameter of the test cell is 20 μm and the pore of the porous material is circular, the diameter of the porous material has only to be extremely smaller than 20 μm, for instance, may be about 0.5 μm. As for the possible size of the test cell other than the above instance, the minimal size is several micrometers, and the maximal size is several tens of micrometers. Accordingly, the porous material can have the average pore size (the average diameter when the pore is circular or the average value of the longest part when the pore has another shape than a circular shape) in a value of 1 μm or smaller, and desirably have the optimal value selected from among values of 1 μm or smaller.

A base material of a porous material is selected from among a material having excellent formability, a material to which sterilization treatment can be applied, and a material having low cytotoxicity. More specifically, the base material includes: a synthetic polymer such as cellulose, polyethylene, polypropylene, nylon, polyester, polyacrylamide and a fluorine resin; an inorganic material such as glass, alumina and titania; and a metallic material such as gold, titanium and stainless steel.

The form of a porous material to be used is arbitrarily selected from among an arbitrary pattern that can be formed through a micromachining processing technique such as a photolithography, a granular form, a fibrous form, a nonwoven fabric form and a sponge-shaped porous body form. The porous material has thus various forms, and may be also a commercially available membrane filter. For instance, a membrane filter purchased from Millipore corporation can be used.

This porous material can be arranged in a flow path so as to traverse the flow path and make its plane direction perpendicular to the flow direction of a solution. A structure is considered for such a flow path, for instance, as to sandwich the porous material between upper and lower substrates that compose the flow path.

(Opening/Closing Section for Flow Path)

When a cell culture treatment device according to the present invention collects cells held by a porous material, the device collects cells from a cell collection port in a different position from a solution inlet, by reversing the flow direction of a fluid toward the porous material. In order to satisfy the above described characteristics, the cell culture treatment device has, for instance, a valve mechanism (opening/closing section for flow path) in the flow path. When the cell culture treatment device has the valve mechanism arranged in the flow path, the device can control the flow direction of the fluid in an appropriate timing when culturing/treating cells and collecting the cells. This opening/closing section for the flow path is arranged in a part at which the cell collection port is connected to the flow path (for instance, a part of the flow path closer to the solution inlet than a dotted line part 21 in FIG. 2B (a part in between a first end and the part 21 at which the cell collection port is connected to the flow path)).

A type of a valve mechanism to be arranged in a fine flow path is not limited in particular, but a desired technique can be appropriately selected from preexisting techniques and be used. A typical valve mechanism reported so far includes a mechanism containing a microactuator, a mechanism using a stimulus-responsive polymer, a mechanism using the surface free energy in the flow path, or a mechanism using a valve.

The mechanism containing a microactuator uses a micromachine produced by using a microprocessing technology. The detail is described in “Technology and Application of Micro Chemical Chip” (Maruzen Co. Ltd.,). Specifically, the micromachine is broadly divided into a diaphragm structure having a partition structure made of a film of which the circumference is fixed, a structure having a diaphragm and a protrusion shape engageable with the diaphragm combined, and a structural silicon substrate having a beam such as a cantilever beam and a doubly supported beam. A mechanism for opening/closing a valve has a structure for blocking a flow path by deforming a membrane provided in the flow path with some type of a driving force, or actuating a valve arranged in the flow path.

A base material which can be used in an opening/closing section for a flow path includes a silicone rubber, a photoresist and a metal. The opening/closing section having a diaphragm or a pillar shape is formed by processing the above described material with the use of a micromachining technology. Main driving force to be used for operating a microactuator includes electrostatic force, electromagnetic force, a piezoelectric element, cubical expansion, a bimetal or an article employing a shape memory alloy.

There is another example of using a stimulus-responsive polymer. A representative stimulus-responsive polymer includes a photoresponsive polymer causing phase separation in response to light, and a temperature-responsive polymer causing phase separation in response to a temperature change.

Particularly, the temperature-responsive polymer can be used because it can be easily controlled and gives small influence on a cell. The temperature-responsive polymer to be used in the present invention may be either a homopolymer or a copolymer. The temperature-responsive polymer presents a high hydrophilic property, is changed into a swollen hydrogel and increases its volume when cooled to a boundary temperature of the polymer or lower, and thereby turns a flow path into a closed state (close). When being placed in the temperature or higher, the temperature-responsive polymer presents weak hydrophobicity and decreases its volume to turn the flow path into an opened state (open).

The specific temperature-responsive polymer can be selected from among a (meth) acrylamide-based compound containing acrylamide or methacrylamide, a (meth) acrylamide derivative containing a cyclic compound such as morpholine, and a vinyl ether derivative such as methyl vinyl ether.

As for a method of coating an applicable wall surface in a flow path with a stimulus-responsive polymer, there are a method of applying the stimulus-responsive polymer to the wall surface, a method of connecting the wall surface to the stimulus-responsive polymer by a chemical reaction, and a method of using a physical interaction. The methods can be singly or concomitantly used. Specifically, the method of connecting the wall surface to the stimulus-responsive polymer by the chemical reaction can employ an electron irradiation technique, a gamma-ray irradiation technique, an ultraviolet irradiation technique, plasma treatment, corona treatment and the like. In addition, when the wall surface and the stimulus-responsive polymer have an adequate reactive functional group, a generally-used organic reaction such as radical reaction, anionic reaction and cationic reaction can be used.

A predetermined region in the flow path can be coated with the stimulus-responsive polymer after having finished setting up the flow path, but the desired region can be efficiently coated by coating the predetermined region on a substrate with the reactive functional group and then finishing setting up the flow path. As for a method of coating the wall surface with the stimulus-responsive polymer by using a physical interaction, there is a method of using physical adsorption force, such as applying the wall surface with the coating material singly or while using a matrix having excellent compatibility with a support as a medium, and mixing the wall together with the coating material or the medium containing the coating material. Such a medium of the matrix includes, for instance, a graft polymer, a block polymer and the like of a monomer forming the support or a monomer having the excellent compatibility with the support, and the coating material.

When a temperature-responsive polymer is used as a stimulus-responsive polymer, an electrothermal conversion body for converting an electric signal to heat, such as a micro-heater, can be used as unit for applying a stimulus to the polymer. Such an electrothermal conversion body is not limited in particular, as long as it is a structure made from a material having higher conductivity than a material around it. For instance, an electrothermal conversion body can be used which is provided with a heat element made from a metal, an alloy or a metallic compound selected from among gold, platinum, chromium, titanium, and ITO (indium-tin-oxide).

The heat element can be formed by a widely known method, for instance, a sputtering method, a vacuum deposition method or a plating method. Such an electrothermal conversion body may be arranged in or outside the pore, or even on the surface of a substrate, and may be embedded in the substrate.

Here, suppose that gold is selected as the material, for instance. Then, because gold has a weak bonding strength to the substrate, a thin film of a metal such as chromium, titanium and tungsten is formed on the substrate so as to improve the bonding strength between the two materials, and then a gold film is formed thereon with a sputtering method. An electrode can be formed by using another method such as a photolithography method and a lift-off method, which is used for generally forming an electrode. When the electrode is formed by using a printed circuit board method or is similarly combined with a Peltier element of an element for temperature control, the electrode can be used as an electrothermal conversion body for setting an arbitrary region on the substrate to a predetermined temperature.

In addition, as another example, there is a method of using the inner surface of a flow path as a valve, by partially changing the surface free energy. The method of changing the surface free energy is to change the wettability of a base material in itself, which can be realized by hydrophobizing or hydrophilizing the surface of the base material.

Specifically, a fluid is an aqueous solution in the present invention, so that a hydrophilic surface enables the aqueous solution to flow thereon more easily and a hydrophobic surface enables the aqueous solution to flow thereon more hardly. Treatment for hydrophilizing the surface of a substrate includes, for instance: a method of modifying the surface of the substrate by introducing a silane coupling agent having a polyethylene glycol chain or a hydroxyl group in an end into the substrate; and the treatment of exposing a silanol group by irradiating the substrate with ultraviolet rays or ozone plasma, or treating the substrate with sulfuric acid. On the other hand, the treatment of hydrophobizing the surface of the substrate includes, for instance: a method of modifying the surface of the substrate by introducing a silane coupling agent to the surface, which contains an alkyl group or a fluorine atom such as a trifluoromethyl group, in the end group; and a method of increasing water-repellency by the surface processing of forming a fine uneven pattern shape with a nanometer to micrometer level on the surface of the substrate with the use of an anodic oxidation technique for silicon.

Further another example is a method of passing a liquid by intentionally forming bubbles in a flow path. The method of forming the bubbles includes a method of using the volume expansion of a gas due to heat and a method of electrochemically generating a gas. Any method can be realized by arranging an electrothermal conversion body and an electrode element in the flow path, which are prepared by using a metallic material as a base material with the above described method.

### Exemplary Embodiments

In the next place, exemplary embodiments according to the present invention will be described, but the present invention shall not be limited to the exemplary embodiments.

### EXAMPLE 1

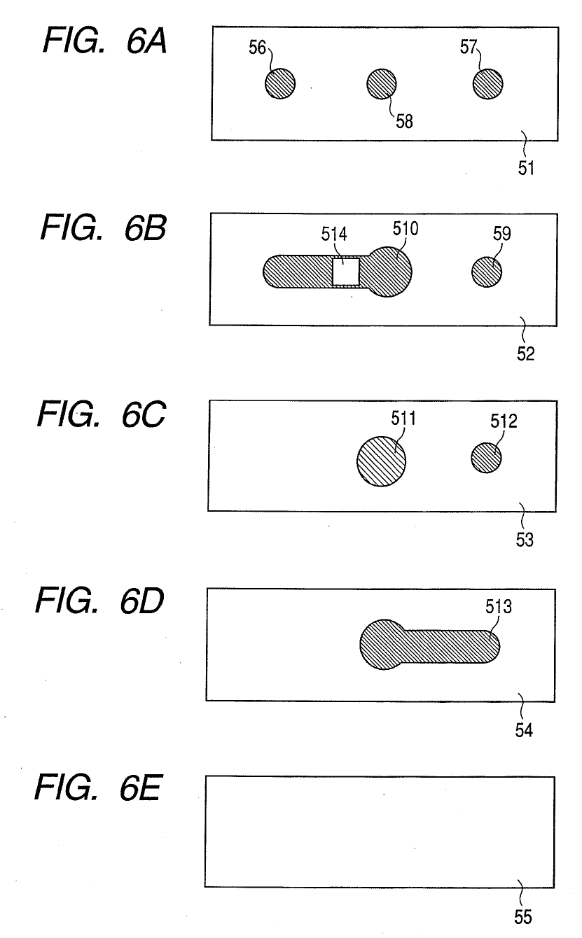

A cell culture treatment device according to the present invention includes a flow path for passing a solution and a porous material for capturing cells. FIG. 5 illustrates one exemplary embodiment of a structure of the cell culture treatment device according to the present invention, and FIG. 6A to FIG. 6E illustrate five sheets of substrates 51 to 55 which compose the cell culture treatment device illustrated in FIG. 5 respectively.

A base material to be used in the present exemplary embodiment can employ an insulating material such as glass, silicon, plastics like polystyrene and a silicone-based elastomeric polymer. A substrate to be used for composing a flow path has a thickness of about 0.2 to 1.0 mm.

The substrate may be mechanically prepared by using a cutting tool such as a drill and a laser. Alternatively, the substrate can be prepared from an elastomer such as polydimethylsiloxane (PDMS), by using a photoresist pattern having a film thickness of several tens of micrometers or more formed with a photolithography method and a versatilely-used metallic pattern, as a mold.

A substrate 51 has a through-hole 56 to be an admission port for a cell-containing liquid and a chemical agent (solution inlet), a through-hole 57 to be an exhaust port (solution outlet), and a through-hole 58 for collecting cells which have been cultured and treated (cell collection port), formed therein respectively. This through-hole 58 is connected to a flow path at a position between a porous material 511 and a part at which the admission port 56 is connected to the flow path. In addition, a lid which can open/close the through-hole 58 is placed on the through-hole 58. A liquid-sending device is arranged at the through-holes 56 to 58. A type of the liquid-sending device is not limited in particular, but is selected from a syringe pump and a peristaltic pump, for instance.

A substrate 52 has a fine flow path groove 510 which has a width of 1 mm or less and a depth of 500 μm or less and is used as a flow path for a liquid sample, and a through-hole 59 for forming the flow path leading to an exhaust port formed therein. A microvalve mechanism (opening/closing section for flow path) 514 is provided at a position in the flow path groove 510, which is closer to an admission port 56 than a part where a cell collection port is connected to the flow path. A microvalve in the device according to the present exemplary embodiment can employ those driven by a piezoelectric element, driven by an electrode, or driven by air sent from a compressor which is placed outside, as previously described. In addition, a microvalve using the dilatation or phase change of a fluid caused by heating can be used.

A substrate 53 has a porous material 511 of a cell-retaining section and a through-hole 512 for forming a flow path leading to an exhaust port formed therein respectively. The cell-retaining section is formed so as to have a diameter of about 1 mm which is equal to or larger than the flow path width. The porous material to be used in the device according to the present exemplary embodiment has only to be able to hold cells and have such a sufficiently large pore size and void as an aqueous solution including a culture medium or a reagent passes without being hindered. In addition, a commercially available material can be used as long as it satisfies the purposes described in claims.

A substrate 54 has a groove 513 for connecting a flow path with an exhaust port formed therein. A substrate 55 is used for forming the bottom surface, and is necessary when a groove formed in the substrate 54 is a through-hole. The substrate 55 is not necessary for facilitating the preparation of the cell culture treatment device, but can be adopted for the purpose of improving the handleability.

The above described substrates are overlapped and adhesively bonded to form a flow path. In the present exemplary embodiment, polydimethylsiloxane (PDMS: sylgard 184, Dow Corning) was used for the substrate 52, and a slide glass (Matsunami) was used for all of the other substrates.

The pattern of a slide glass was formed by masking the slide glass with a metallic sacrificial film and with the use of a photolithographic technique and wet-etching the slide glass with hydrofluoric acid. A PDMS structure (elastomer) which is sandwiched between the slide glasses in the present exemplary embodiment was prepared by forming a resist pattern on the slide glass with a photolithographic technique and by transferring the pattern as a mold. In the present exemplary embodiment, the mold was prepared by using a commercially available negative resist (SU-8; MicroChem Corp.).

At first, a precursor of PDMS was charged into the mold and was heated at 90° C. with an oven for one hour in the state of the mold and the prepolymer being sandwiched between the slide glasses, and was solidified into the polymer. The set was radiationally cooled, and the PDMS was removed from the mold to provide a PDMS elastomer. In order to facilitate the removal of the PDMS elastomer from the mold in the above step, the surface of the mold may be treated with a silane coupling agent such as 3, 3, 4, 4, 5, 5, 6, 6, 6-Nonafluorohexyltrichlorosilane (Shin-Etsu Chemical Co., Ltd.) in advance, so as to make the surface water-repellent.

Incidentally, when the PDMS elastomer is used as a base material, the substrates can be spontaneously bonded to each other. The PDMS elastomer can be bonded to the slide glass by melting the PDMS elastomer with oxygen plasma (80 W, 30 seconds).

As for methods for bonding other substrates, for instance, when glass is selected as a base material, the substrates can be bonded by any one selected from among a hydrofluoric acid solution, a spacer which is generally selected from a high polymeric material such as glass and teflon, and a silicone-based adhesive. However, the adhesive is not limited in particular, as long as it is such a material as is not eroded by a passing solution. The substrate can be bonded more strongly by appropriately using a weight.

In the present device, a porous material made for cellulose acetate was used, which is commercially available from Millipore Corporation. The porous material used in the present device was selected so as to have a suitable pore size in consideration of a size of a cell body and a condition capable of satisfactorily sending a solution. When cells of which the single cell has a large size are used, for instance, the porous material having an average pore size of 10 μm or smaller can be used, and furthermore, a porous material having an average pore size of 1 to 5 μm can be used.

In the present exemplary embodiment, a slide glass and a PDMS elastomer were used for a base material as described above. The PDMS elastomer was selected for the base material of a substrate 52, because of generally having higher water-repellency than the slide glass, and was used as a valve which makes use of the water-repellent force.

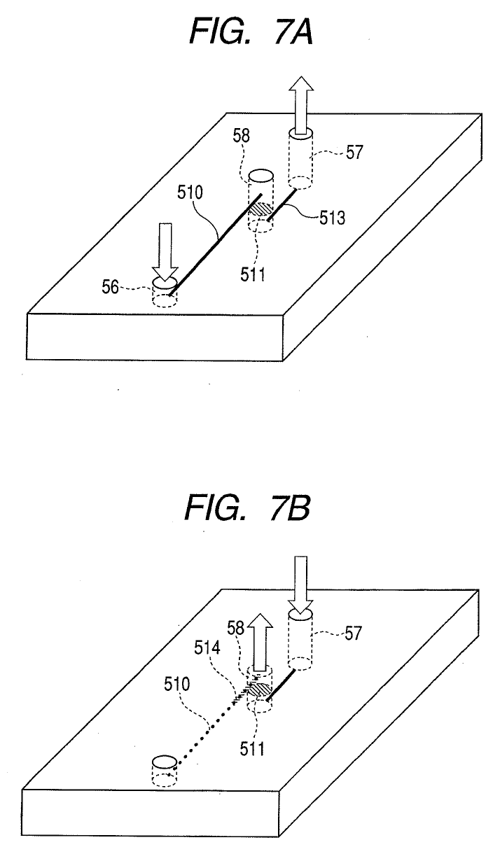

Specifically, when cells are cultured as usual, an outlet 58 which is a cell collection port for the cells is closed by a lid. Then, a liquid introduced from a solution inlet 56 passes through a flow path 510 and a cell-retaining section 511 and is drained from the solution outlet 57 (FIG. 7A). Subsequently, the cells are cultured or treated for a desired period of time, and then the outlet 58 is opened. When the liquid is sent in a reverse direction, desired cells can be easily collected through the cell collection port 58 (FIG. 7B), because the surface of a region 514 has higher water-repellency than that of regions around the region 514.

As for an device for changing a liquid-sending direction in the above step, it is desirable to attach a push pull pump to an outlet 57 side and switch a liquid-sending direction. However, the liquid can be sent in different directions between a liquid-sending time for culturing cells and a liquid-sending time for collecting the cells, by changing the position for the pump to be attached. In the present exemplary embodiment, a push-pull-type syringe pump (Harvard Apparatus) was used.

### EXAMPLE 2

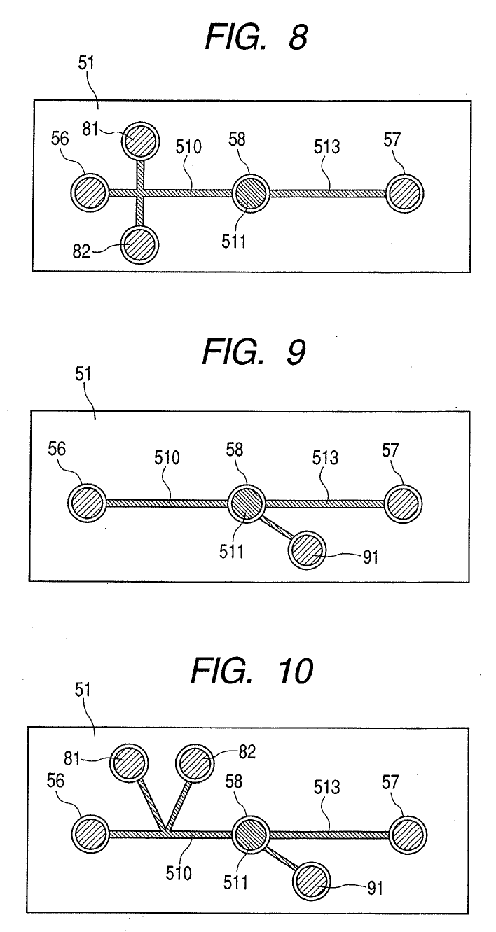

A cell culture treatment device according to the exemplary embodiment was produced with the same method as in the case of Example 1, except that three admission ports 56, 81 and 82 for a chemical agent were arranged (FIG. 8). Specifically, this cell culture treatment device has three solution inlets which communicate with a flow path, and can properly use the three solution inlets for different purposes according to a type and amount of a solution to be used (cell-containing liquid, culture medium, reagent and the like), or simultaneously use some of them. For instance, a cell-containing liquid is sent from the first solution inlet, and cells are retained in a cell-retaining section 511. Then, the supply of the cell-containing liquid is stopped, and at the same time, the culture medium is sent from the second solution inlet. Thereby, the cell culture treatment device can continuously apply a pressure of the flow to the cells, and can make the cell-retaining section effectively retain the cells.

### EXAMPLE 3

A cell culture treatment device according to the exemplary embodiment was produced with the same method as in the case of Example 1, except that two cell collection ports 58 and 91 were arranged (FIG. 9). Specifically, this cell culture treatment device has two cell collection ports which communicate with a flow path, and can properly use the two cell collection ports for different purposes according to a type and amount of a cell to be collected, or simultaneously use them.

### EXAMPLE 4

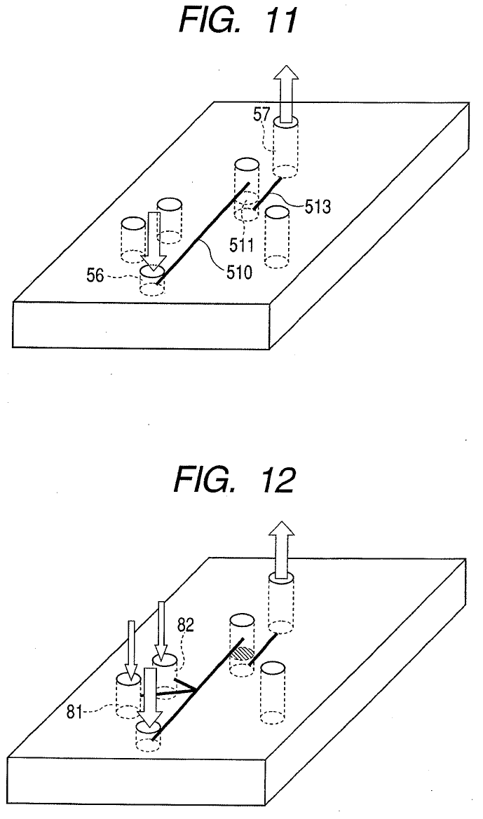

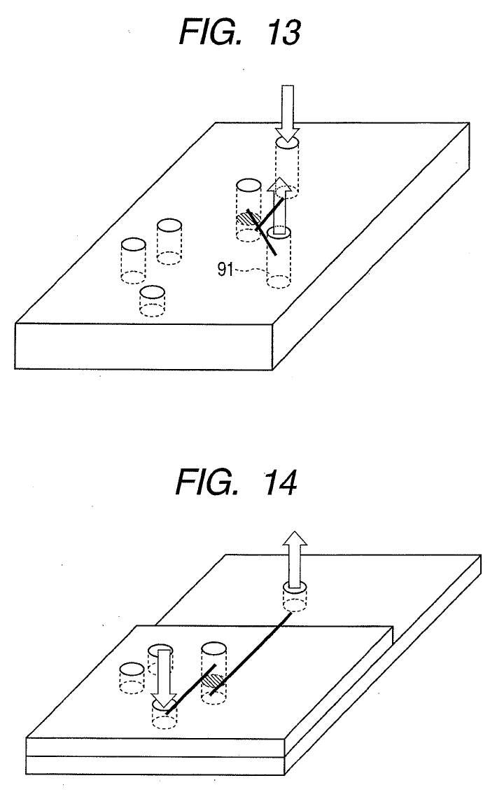

A cell culture treatment device according to the exemplary embodiment was produced with the same method as in the case of Example 3, except that three solution inlets 56, 81 and 82 were arranged (FIG. 10). By using the device provided with three solution inlets as in the case of the exemplary embodiment, such operations can be collectively performed as passing a solution to a flow path through the solution inlet 56 (FIG. 11) and introducing a chemical solution through the solution inlets 81 and 82 (FIG. 12). In addition, the device can collect cells through a cell collection port 91 (FIG. 13).

### EXAMPLE 5

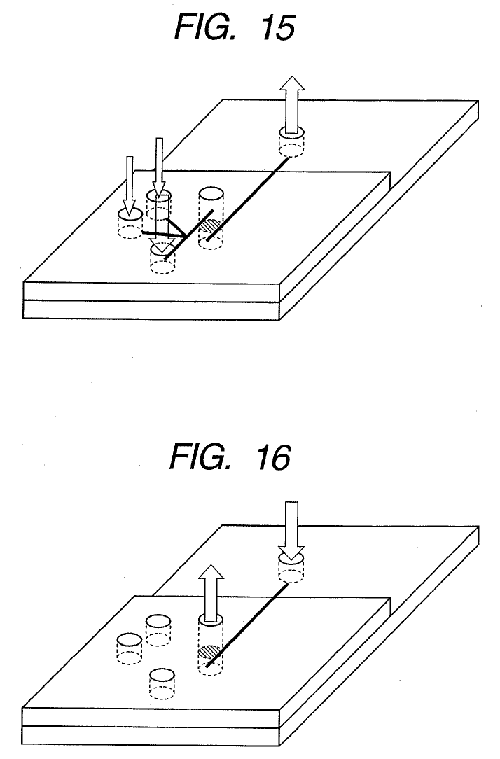

A cell culture treatment device according to the exemplary embodiment was produced with the same method as in the case of Example 1, except that the height of a solution outlet is different from the other ports. When the device provided with three solution inlets as in the case of the exemplary embodiment is used, the device can pass a liquid to a flow path (FIG. 14), introduce a chemical solution (FIG. 15), and collect cells (FIG. 16).

### EXAMPLE 6

A perfusion culture experiment for animal cells was conducted by using a cell culture treatment device produced in a method of Example 1. The cell culture treatment device in the present exemplary embodiment employed cellulose acetate having the pore size of 3 μm for a porous material. The cell culture treatment device was previously subjected to the sterilization treatment of irradiating the device with UV rays. Cells used in the exemplary embodiment were HepG2 cells which were human-hepatic-cancer-derived cells.

The used HepG2 cells had been previously subcultured for third to fifth passage, on culture conditions of 37° C. and 5% CO₂ in a cell culture flask (Falcon). The HepG2 cells were detached from the bottom surface of the culture flask by treating the cell suspension with the enzyme of trypsin. The concentration of the cells was adjusted to 5.0×10⁶ cells/mL with the use of a hemocytometer.

A used cell culture medium was a Dulbecco's modified Eagle's medium (DMEM; INVITROGEN) containing 10% bovine serum and a high content of glucose. FIGS. 7A and 7B illustrate a schematic view of a cell culture operation.

In the exemplary embodiment, a liquid was sent by using a syringe pump. At first, a cell collection port 58 was closed, and a cell suspension (cell-containing liquid) was passed from a solution inlet 56 to a solution outlet 57 at a flow rate of 5 μL/min for 10 minutes by using the syringe pump. After the suspension had been sent, the syringe pump was stopped (FIG. 7A). In this step, cells were retained in a cell-retaining section 511 (retaining step). Subsequently, a syringe for introduction was exchanged and a DMEM culture medium was introduced into a flow path. The medium was continually sent to the solution outlet 57 from the solution inlet 56 at a flow rate of 5 μL/min for 72 hours to have cultured the cells (culture step).

The cells were cultured in an incubator kept at 37° C., and the culture medium introduced into the flow path had a mixture gas including oxygen, carbon dioxide and nitrogen adjusted to 10%, 5% and 85% respectively blown therein. After the cells had been cultured for a predetermined period of time, the cell collection port 58 was opened, the solution was passed to the cell collection port 58 from the solution outlet 57 as illustrated in FIG. 7B to liberate the cells from the cell-retaining section 511, and the cells were collected in the cell collection port 58 (collecting step).

The collected cells were further subjected to the observation of the form with the use of a microscope, or the evaluation of a cell function such as an albumin production amount, with the use of a commercially available kit. The amount of albumin produced by HepG2 cells in the culture with the use of the cell culture treatment device according to the present invention was compared to that produced by the HepG2 cells statically cultured in a cell culture flask, and as a result, the HepG2 cells cultured in the cell culture treatment device showed an albumin synthesis capability equivalent to or better than those cultured statically. From the above described result, it is understood that the cell culture treatment device in the exemplary embodiment works as a three-dimensional culture device in which hepatic cells agglomerate with each other, because the HepG2 cells are cultured without bonding to the porous structure.

### EXAMPLE 7

Escherichia coli with extremely high chemotaxis was cultured as in the case of Example 6 except that an Escherichia coli cell body (Escherichia coli K12 strain) was used in place of a HepG2 cell. A medium of Trypto-Soya Agar (NISSUI PHARMACEUTICAL CO., Ltd.) was used for culturing K12.

Escherichia coli was cultured on an agar medium on conditions of 38° C. and 12 hours and was collected. A suspension (cell-containing liquid) was prepared by suspending the collected Escherichia coli in a liquid culture medium (YT culture medium). The number of the bacteria in the medium was adjusted to 1.0×10⁸ bacterias/mL. The cell body was cultured, collected and observed with the use of Double Staining Kit (DOJINDO LABORATORIES). As a result of having observed a ratio of Live/Dead of the cell bodies, it was confirmed that 90% or more of the cell bodies survived. From the result, it became clear that Escherichia coli cell body can be cultured with the use of the cell culture treatment device according to the present invention.

### EXAMPLE 8

Free-floating cells were cultured with the same method as in the case of Example 6 except HL60 cells of hematocyte cells were used in place of HepG2 cells.

### EXAMPLE 9

Cells were cultured on microcarriers of fine particles while using the device used in Example 6. A microcarrier bead used in the present exemplary embodiment was a bead commercially available (diameter of 0.1 mm) from Pharmacia or the like. A cell used for the culture was a CHO cell (Chinese hamster utero-ovary cell). A medium used for the culture was an e-RDF culture medium (KYOKUTO PHARMACEUTICAL INDUSTRIAL CO., Ltd.), which contained 10% fetal bovine serum. The microcarrier beads were sterilized and prepared into a suspension adjusted to the concentration of 5 g/L. The suspension was mixed with a cell suspension (cell-containing liquid) adjusted to 5×10⁶ cells/ml. The mixed suspension was statically cultured in a cell culture flask for 24 hours to make the cells bond to the surface of the bead.

Microcarrier bead on which cells bonded were collected from a flask, were introduced into the device used in Example 6 through a solution inlet, and were retained in a cell-retaining section 511. The cells were cultured on the microcarriers while the culture medium was sent to the cell-retaining section.

The culture medium was sent to the cell-retaining section at a flow rate of 5 μL/min for five days. Subsequently, the amount of the serum added to the culture medium was gradually reduced and the culture medium was switched into a serum-free medium before the tenth day. The cells were further cultured for 20 days while the serum-free medium was circulated in the flow path. The cultured cells were collected together with all the beads by changing a liquid to be sent.

The shape and density of the cells on the surface of the collected beads were measured through a fluorescence microscope by using Double Staining Kit (DOJINDO LABORATORIES). The cells were concentrated onto the bottom surface with a centrifugation operation, and were further suspended again in a predetermined quantity of sterilized water. Then, the turbidity of the suspension was measured. As a result of having measured the cells with the fluorescence microscope and the turbidity of the suspension, it was confirmed that the cells survived and bred. From the result, it was shown that the device according to the present invention is effective for a microcarrier culture.

### EXAMPLE 10

Modified CHO cells were cultured on microcarriers with the same method as in Example 9 except that the modified CHO cells were used to which the ability of producing a granulocyte colony-stimulating factor (G-CSF) was hereditarily imparted.

By using a cell culture treatment device according to the exemplary embodiment of the present invention described above, only the cells which have been cultured for a desired period of time or treated in various ways can be easily and efficiently collected in a short time.

In addition, the cell culture treatment device according to the exemplary embodiment of the present invention has a cell-retaining section made from a porous material or the like arranged in a flow path, and can capture cells in the cell-retaining section made from the porous material or the like provided in the flow path. Accordingly, the cell culture treatment device can reduce an influence of stacking of the cells retained in the cell-retaining section; can efficiently culture the cells; and can easily exchange a culture medium.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-232178, filed Aug. 29, 2006, which is hereby incorporated by reference herein in its entirety.

What is claimed is:

1. A cell culture treatment device for culturing, treating and collecting cells, comprising:
a flow path for passing a cell suspension, a chemical solution and a buffer solution;
a cell-retaining section which can retain cells and is placed in the flow path;
a solution inlet connected to a first end of the flow path;
a solution outlet connected to a second end of the flow path;
a cell collection port connected to the flow path in between the cell-retaining section in the flow path and the first end; and
a lid capable of opening/closing the cell collection port.

2. The cell culture treatment device according to claim 1, wherein the cell-retaining section is made from a porous material.

3. The cell culture treatment device according to claim 2, wherein the flow path includes a flow path which extends in a vertical direction, and the porous material is arranged in the vertical flow path so that the plane direction can be parallel to the bottom part of the flow path.

4. The cell culture treatment device according to claim 2, wherein the porous material has an average pore size smaller than a size of a single cell.

5. The cell culture treatment device according to claim 2, wherein the average pore size of the porous material is 1 μm or smaller.

6. The cell culture treatment device according to claim 1, further comprising an opening/closing section for the flow path, which can open/close the flow path and is arranged at a portion in the flow path between the first end and a part at which the cell collection port is connected to the flow path.

7. The cell culture treatment device according to claim 1, further comprising a liquid-sending unit which can send a solution and is connected to at least one of the solution inlet and the solution outlet.

8. A cell culture treatment method with the use of the cell culture treatment device according to claim 1, comprising the steps of:
passing a cell suspension containing the cells from the solution inlet to the solution outlet with the cell collection port closed with the lid to make the cell-retaining section retain cells,
performing at least one of the culture of the cells and the treatment of the cells after the retaining by passing from the solution inlet to the solution outlet at least one solution selected from the group consisting of a cell culture medium, a chemical solution for treating the cells and a buffer solution in the state of making the cell-retaining section retain the cells; and
flowing a solution from the solution outlet to the cell collection port after the performing step with the cell collection port opened with the lid to liberate the cells which have been retained in the cell-retaining section and collect at the cell collection port the cells.

9. A cell culture treatment method with the use of the cell culture treatment device according to claim 6, comprising the steps of:
passing a cell suspension containing the cells from the solution inlet to the solution outlet with the cell collection port closed with the lid and the opening/closing section opened to make the cell-retaining section retain cells,
performing at least one of the culture of the cells and the treatment of the cells after the retaining by passing from the solution inlet to the solution outlet at least one solution selected from the group consisting of a cell culture medium, a chemical solution for treating the cells and a buffer solution in the state of making the cell-retaining section retain the cells; and
flowing a solution from the solution outlet to the cell collection port after the performing step with the cell collection port opened and the opening/closing section path closed with the lid to liberate the cells which have been retained in the cell-retaining section and collect at the cell collection port the cells.
